(19) United States
(12) Patent Application Publication
(10) Pub. No.: US 2018/0257217 A1
(43) Pub. Date: Sep. 13, 2018

(54) LOAD-BEARING EXOSKELETON

(71) Applicant: The Regents of the University of Michigan, Ann Arbor, MI (US)

(72) Inventors: Daniel D. Johnson, Ann Arbor, MI (US); Hitinder S. Gurm, Ann Arbor, MI (US)

(21) Appl. No.: 15/756,467

(22) PCT Filed: Sep. 2, 2016
(86) PCT No.: PCT/US16/50077

§ 371 Date: Feb. 28, 2018

Related U.S. Application Data
(60) Provisional application No. 62/214,401, filed on Sep. 4, 2015.

(51) Int. Cl. B25J 9/00; A61F 5/01; B25J 19/00

(52) U.S. Cl. CPC B25J 9/0006; A61H 3/00; B25J 19/0016; A61H 2203/0406; A61F 2005/0167; A61F 2005/0158; A61F 2005/0169; A61F 2005/0197; A61H 2201/0107; A61H 2201/0165; A61H 2201/0176; A61H 2201/1207; A61H 2201/1614; A61H 2201/1619; A61H 2201/1628; A61H 2201/164; A61H 2201/165; A61H 2201/1676; A61H 2201/169; A61F 5/0123

(57) ABSTRACT

A load-bearing exoskeleton capable of supporting at least part of the weight of a shielding garment. Additionally, some of the user's upper-body weight, resulting from the rotational moment about the hip caused by the user's trunk in flexion, may be supported. The load-bearing exoskeleton may be a completely passive orthosis, or it may include one or more active orthotic elements. The exoskeleton may include one or more sagittally-extending load-bearing structures that provide a supportive force to counteract at least some of the weight of the shielding garment. The exoskeleton may include a shielding garment attachment mechanism, a pelvis assembly, and one or more leg assemblies that are configured to allow for user movement when in one or more unlocked positions, while facilitating a greater transmission of weight of a shielding garment when in one or more locked positions.

### CROSS-REFERENCE TO RELATED APPLICATION

This application claims the benefit of U.S. Provisional Application No. 62/214,401 filed Sep. 4, 2015, the entire contents of which are hereby incorporated by reference.

### TECHNICAL FIELD

This invention relates generally to exoskeletal devices, and more particularly to load-bearing exoskeletons that can support weight.

### BACKGROUND

Back pain is a common occupational injury, which can lead to lost productivity and significant expenditures of medical resources annually. Back pain is often associated with occupations requiring frequent bending and lifting maneuvers, which can impose considerable loads on the spine. While large loads increase the risk for injury, sustained static flexion of the spine while supporting the weight of the trunk alone can also lead to back pain as the extensor muscles of the lower back become fatigued. Similarly, prolonged awkward postures of the head and neck can produce discomfort.

During various treatment procedures, physicians are often required to adopt sustained static flexion of the spine. The performance of physicians in the operating room can be adversely affected by postural fatigue and discomfort, which are aggravated by the static postures frequently required during procedures. General surgeons, for example, can spend 65% of their operating time in static postures of the head and neck, with 14% of those in a flexed (forward bent) position. Physicians who perform minimally-invasive (e.g., laparoscopic, endoscopic, etc.) surgical procedures also experience long periods of static postures.

One subgroup of operating physicians that is believed to experience a higher-than-average incidence of back pain is interventionalists. These include neurosurgeons, radiologists, and cardiologists, for example, who operate using real-time radiography. The radiation levels in the operating room require the use of shielding garments (also called “leads”) for the full duration of procedures. The added weight of these garments on the trunk can potentially increase the risk for neck, shoulder, and/or back pain. One study showed that physicians who used shielding garments regularly (in this case, cardiologists who wore leads up to 8.5 hours per day) had the highest incidence of missed work days due to neck/back pain (21.3%) and required more treatment than other physicians who did not have to use shielding garments. The same study also showed a higher incidence of multiple-disc herniations of the cervical and lumbar spine among interventionalists. Approximately 20% of interventional cardiologists will develop symptoms of invertebral disc degeneration, and about 5% will require surgical intervention to treat the condition, which typically requires 22 days or more of recovery. Moreover, because the activity of the lower back muscles is known to directly correlate with lumbar intervertebral disc pressure, prolonged exposure to high intervertebral pressures, such as when a shielding garment is worn, can lead to discomfort as well as permanent structural damage of the intervertebral discs.

Physicians often employ a variety of creative methods to try and mitigate discomfort, including the use of spinal orthotics worn under shielding garments and surgical gowns. Spinal orthotics such as soft belts and semi-rigid corsets that are currently available can achieve some degree of spinal offloading by increasing intraabdominal pressure as well as serving as a kinesthetic reminder to the user to prevent excessive flexion. However, it has been shown that the use of such commercially-available back belts provides no reduction in the likelihood of injury, as quantified through compensation claims and reported lower back pain. Custom-made orthoses produced by a trained orthotist have been shown to be more biomechanically effective than common mass-produced, non-customized, or over-the-counter models, but have several drawbacks: the individual manufacturing and fitting required are prohibitively expensive for common usage, the restricted maneuverability such orthoses create could be disadvantageous in the workplace, and the increased back postural muscle activity that some orthoses can produce could actually promote muscle fatigue.

Aside from using over-the-counter back braces or one-off solutions created by individual clinicians, there have been a number of products that attempt to offload the weight of shielding garments. One includes a mobile scaffold that suspends the shielding garment over its user. The device must be wheeled about the operating room by two handles at waist level. Another suspended radiation protection system can carry the shielding garment and a face shielding window array via an overhead arm fixed to the ceiling of the room. Other repositionable shields can be rolled around or mounted to arms fixed to a wall of the operating room. These devices have not been widely adopted, may be obtrusive in a treatment room or prohibit the physician from certain types or directions of movement, or could be prohibitively expensive.

Shielding garments may also be used for chemical and radiation protection in non-medical scenarios such as nuclear leaks, chemical spills, etc. While most of the work in such scenarios is performed by robots or other machines, it may be desirable to have human participation. Providing a more mobile and low-profile shielding garment support could help facilitate such human contribution in those instances.

### SUMMARY

According to one embodiment, there is provided a load-bearing exoskeleton comprising a plurality of sagittally-extending load-bearing structures. At least one sagittally-extending load-bearing structure is an upper body support and at least one sagittally-extending load-bearing structure is a lower body support. A supportive force is provided to counteract at least some of the weight of a shielding garment. The supportive force is at least partially normal to an outer surface of at least one of the sagittally-extending load-bearing structures when an applied force from the shielding garment is encountered. The applied force from the shielding garment is at least partially transmitted to the floor through the at least one lower body support.

According to another embodiment, there is provided a load-bearing exoskeleton comprising at least one shielding garment attachment mechanism and a pelvis assembly including one or more hip joints. The attachment mechanism is connected to and supported by the pelvis assembly. The load-bearing exoskeleton further comprises one or more leg assemblies attached to the pelvis assembly via the one or more hip joints. The hip joint of the pelvis assembly is configured to allow for rotational movement of the leg assembly about a medial-lateral axis.

According to another embodiment, there is provided a load-bearing exoskeleton comprising at least one shielding garment attachment mechanism and a pelvis assembly. The load-bearing exoskeleton further comprises one or more leg assemblies attached to the pelvis assembly. Each leg assembly includes a rotational joint at a location generally corresponding to a greater trochanter of a user. The rotational joint includes a locking mechanism that is configured to inhibit rotational movement of the leg assembly when the exoskeleton is in a locked position.

### BRIEF DESCRIPTION OF THE DRAWINGS

Preferred exemplary embodiments will hereinafter be described in conjunction with the appended drawings, wherein like designations denote like elements, and wherein:

FIG. 1 is an isometric view of a load-bearing exoskeleton in accordance with one embodiment;

FIG. 2 shows the load-bearing exoskeleton of FIG. 1 on a user with a shielding garment that is attached over the exoskeleton;

FIG. 3 is an enlarged view of one embodiment of a hip joint for a load-bearing exoskeleton;

FIG. 4 is an enlarged view of a mobile internal portion for the hip joint of FIG. 3;

FIG. 5 is an enlarged, partial view of a load-bearing exoskeleton, showing the hip joint of FIG. 3 and the attachment of a leg assembly;

FIG. 6 shows a hip joint for a load-bearing exoskeleton in accordance with another embodiment; and

FIGS. 7A-7C show another embodiment of a knee joint that may be used with various implementations of the load-bearing exoskeleton.

### DESCRIPTION OF THE ILLUSTRATED EMBODIMENTS

A load-bearing exoskeleton as described herein can help to offload the weight of a shielding garment, for example, from the body of a user. Preferably, the load-bearing exoskeleton can offload the entire weight of a shielding garment to help alleviate the risk of back injury and discomfort. The mass of a shielding garment may be borne entirely by the exoskeleton and conveyed down to the floor, with the user providing structural alignment. It is advantageous for the exoskeleton to have a low profile so as to not interfere with tight spaces in the operating room, while still affording sufficient mobility. Embodiments of the load-bearing exoskeleton may allow a user to rotate normally in place (e.g., turn about), walk, and tilt (e.g., flex) the trunk at the waist in one or more body planes. Some embodiments of the load-bearing exoskeleton allow a user to enter from behind, by simply walking into the device and then securing it to his or her body via an attachment mechanism such as adjustable straps or the like. In a preferred embodiment, the load-bearing exoskeleton is customized to a user's unique anthropometry. This customization may be facilitated by the use of easily-scalable computer aided design (CAD) models and three dimensional (3D) printing of complex parts. In another embodiment, each exoskeleton may be tailored to fit multiple end users of a similar body type, so that a customer (e.g., a hospital) does not need to necessarily purchase a unique exoskeleton for each end user.

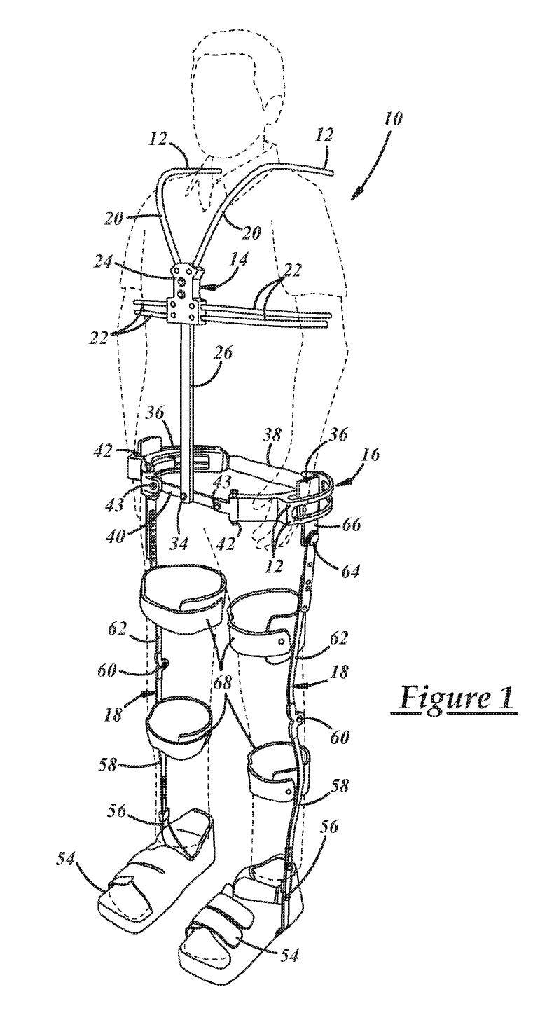

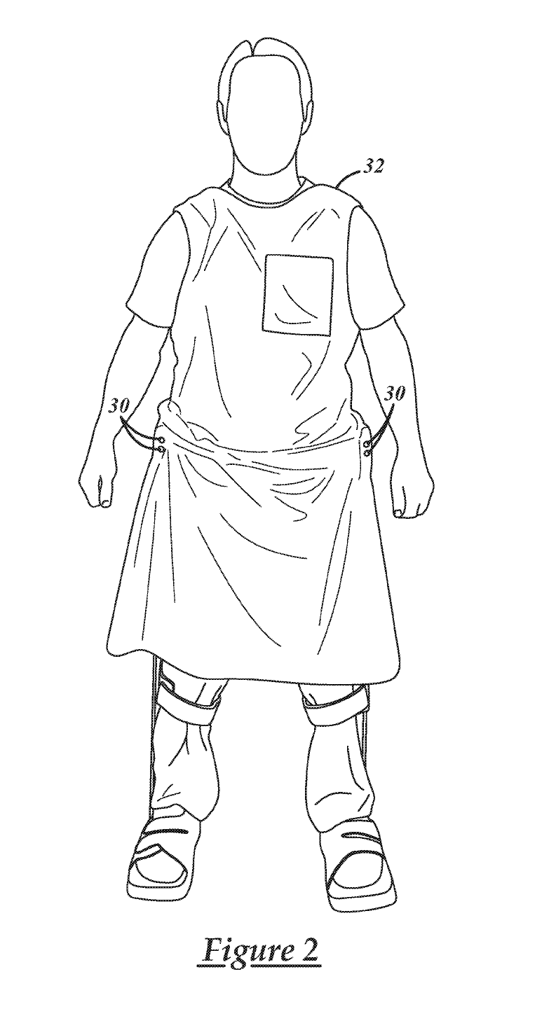

FIGS. 1 and 2 illustrate one embodiment of a load-bearing exoskeleton 10. FIG. 1 shows the load-bearing exoskeleton, and FIG. 2 shows the load-bearing exoskeleton on a user with a shielding garment. The illustrated exoskeleton is a passive orthosis, although it may be possible in some embodiments to include one or more active orthotic elements. This embodiment of the load-bearing exoskeleton 10 has a low profile against the user's body. Such a compact design helps to avoid costly modifications to the operating room that are often required with prior art designs, such as arm-mounted shields that require the installation of overhead load-bearing arms or scaffolding. Additionally, the exoskeleton 10 has the potential to provide desired weight offloading without sacrificing any additional operating room volume, which nearly all prior art devices consume to at least some degree. As will be described in detail below, the load-bearing exoskeleton 10 may be capable of transitioning between locked and unlocked positions, with the unlocked position allowing for nearly natural motion and thereby avoiding the cumbersome nature of nearly all existing solutions. Further, it should be understood that a locked position may be at least partially transitional, so long as the exoskeleton can offload all or part of one or more applied forces. Accordingly, there may be various locked positions, such that the exoskeleton can offload in multiple postures. For example, the exoskeleton may allow for a more natural bend in the knees while standing. Other examples of locked positions are certainly possible. Various embodiments of the load-bearing exoskeleton may include a shielding garment attachment mechanism 12, a thorax assembly 14, a pelvis assembly 16, and/or one or more leg assemblies 18.

Some load-bearing exoskeleton embodiments include at least one shielding garment attachment mechanism 12. In one embodiment, the shielding garment attachment mechanism 12 may be the thorax assembly 14 itself or parts thereof. In the embodiment illustrated in FIGS. 1 and 2, shoulder extensions 20 and rib extensions 22 can radiate from a thoracic nexus 24 and generally extend around the shoulders and ribs of a user, respectively. In one embodiment, the shoulder extensions and/or rib extensions are the shielding garment attachment mechanisms, which may be slid into corresponding sleeves or pockets on the interior of the shielding garment. This embodiment may help to keep the shape of the shielding garment. In another example, pins or another fastening device may be used to attach the shielding garment to a portion of the exoskeleton, such as at shielding garment attachment mechanisms 12, which in this embodiment, are recesses for a plurality of fasteners which are located on the pelvis assembly 16. As shown in FIG. 2, fasteners 30 may serve help attach the shielding garment 32 to the shielding garment attachment mechanisms 12. Other shielding garment attachment mechanisms are certainly possible, such as bolts, reinforcing plates, etc. In one embodiment, the shielding garment attachment mechanisms may include high friction pads on the outer surface of the exoskeleton that can prevent the shielding garment from falling off. The shielding garment itself may be custom-tailored, and it could be a single piece (e.g., a smock-like garment attached to the rib extensions of the thorax assembly) or multiple pieces (e.g., a vest portion from the user's neck to the hips and supported by the rib extensions with a skirt from the user's hips to knees or below that is pinned to the pelvis assembly). In another embodiment, the shielding garment attachment mechanism to could include a shielding garment that is specially modified to be a part of or integrated with the exoskeleton. Additionally, other shielding garment features may be included, such as a discrete vest, a neck collar for enhanced thyroid shielding, variable sleeve lengths, or a special mass distribution (e.g., more mass toward the back to further reduce muscle strain in flexion, by offloading torso weight and the weight of the shielding garment). Further, smaller, larger, or differently shaped shielding garments may be used.

The thorax assembly 14 in this embodiment includes shoulder extensions 20 and rib extensions 22 that radiate from the thoracic nexus 24. In a preferred embodiment, the thorax assembly is open in the back, which allows a user to simply walk into the device. The thoracic nexus 24 is attached to the pelvis assembly 16 via central beam 26. The thoracic nexus 24 can help to shunt weight from a shielding garment, or more particularly, from a trunk-worn portion of a shielding garment such as a discrete vest, into the central beam 26. The central beam 26 in this particular embodiment is a metal bar that is pivotally mounted with a rotational joint 34 to the pelvis assembly 16 to help facilitate lateral flexing of a user's torso. The shoulder extensions 20 or the central beam 26 may be considered a sagittally-extending load-bearing structure. “Sagittally-extending” refers to any position less than orthogonal to the sagittal plane of a user. Each rib extension may serve as a transversely-extending load-bearing structure, with “transversely-extending” referring to any position less than orthogonal to the transverse plane of a user. Sagittally-extending load-bearing structures and transversely-extending load-bearing structures that are part of the thorax assembly may be classified as upper body supports. Similarly, “coronally-extending” may refer to any position less than orthogonal to the coronal plane of a user. Thus, the rib extensions may also at least partially include coronally-extending load-bearing structures. The load-bearing structures can help to provide a supportive force when an applied force (e.g., weight) from a shielding garment is encountered, thereby offloading some of the force from the user. The supportive force may be at least partially normal to an outer surface of the load-bearing structure (e.g., in a direction away from the user), and is distinguishable from prior art devices that hold a shielding garment above a user, for example. In a preferred embodiment, as shown in FIG. 1, the thorax assembly 14 is designed to reside in front of or anteriorly to the user and be supported by the single pivot at the center of the abdomen (e.g., in this embodiment, the rib extensions 22 are designed so as to not rest against the user, although they may enhance alignment with the user's torso). Fabric straps that can be tied at the back of the user when the shielding garment is donned may be used to maintain alignment with the user's torso.

The pelvis assembly 16 is located between the thorax assembly 14 and the leg assemblies 18 in the embodiment illustrated in FIGS. 1 and 2. The pelvis assembly 16 in this embodiment includes two hip joints 36, a strap 38 which connects the two hip joints 36 in the posterior, a metal bar 40 which connects the two hip joints 36 in the anterior via hinges 42 and pelvic rotational joints 43. The strap 38 and hinges 42 can help to provide easy ingress and egress from the exoskeleton 10. Pelvic rotational joints 43 can allow for pelvic tilting while a user is walking in the exoskeleton. The pelvic rotational joints 43, if provided, are preferably positioned near the center of rotation of each hip. The pelvis assembly may include padding, and may partially rest on a user's hips, or it may not rest on a user's hips at all. The pelvis assembly may include one or more transversely-extending load-bearing structures and/or one or more coronally-extending load-bearing structures.

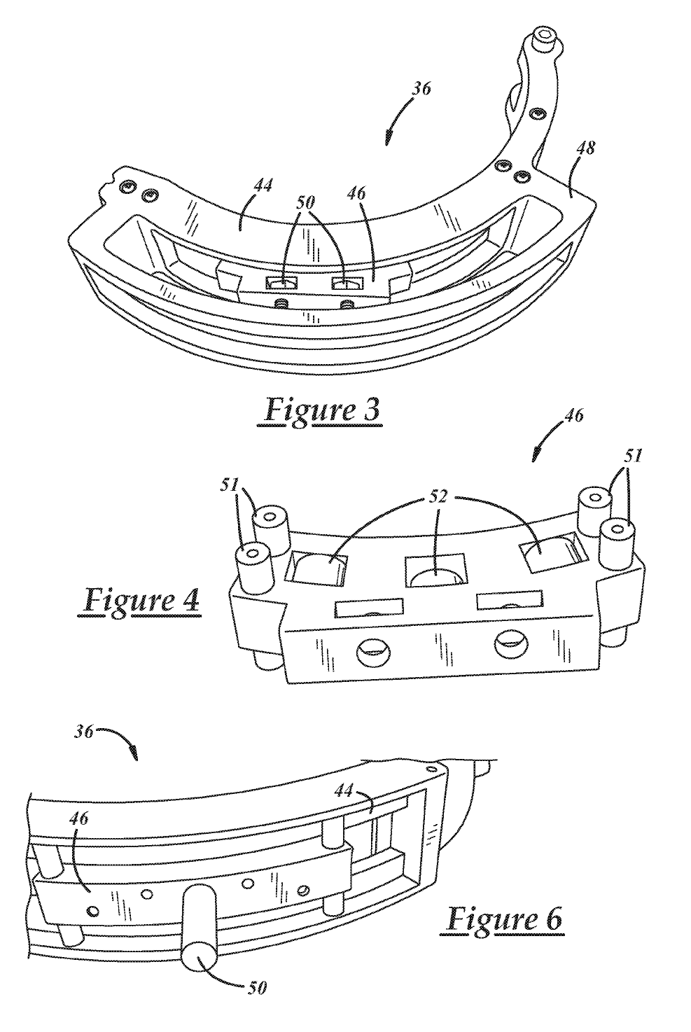

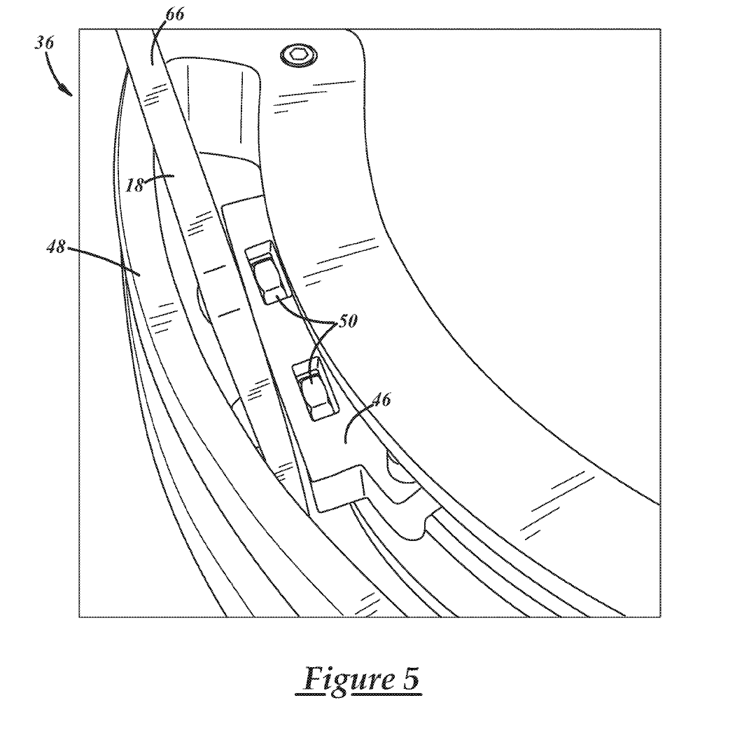

FIG. 3 is an enlarged view of one embodiment of a hip joint 36. The hip joints 36 of the pelvis assembly 16 are configured to allow for rotational movement of the leg assemblies 18 about a medial-lateral axis. Thus, users can rotate their legs in place about a vertical axis, thereby permitting users to turn about in place. This hip joint embodiment includes a track assembly 44 and an internal mobile portion such as a cart assembly 46 that can slide along the track assembly. An outer guard 48 may be provided to help shield the movable portions of the hip joint 36 such as the cart assembly 46, and fasteners 50 which may be used to attach a leg assembly. In one embodiment, the hip joint track assembly and cart assembly are made from printed acrylonitrile butadiene styrene (ABS) plastic. FIG. 4 is an enlarged view of one embodiment of a cart assembly 46. This embodiment of the cart assembly includes stabilizing components 51, which in this implementation, are rollers that help facilitate translation along the track assembly 44. This embodiment of the cart assembly also includes translation components 52, which in this implementation, are rollers that help decrease friction between the track assembly 44 and the cart assembly 46. FIG. 5 illustrates a load-bearing exoskeleton on a model, showing how the leg assembly 18 may be attached via fasteners 50 to the hip joint 36. Other methods of attachment are certainly possible, such as using an adhesive to mount the leg assembly to the cart. FIG. 6 shows another embodiment of a hip joint 36 having a track assembly 44 and a cart assembly 46, but no outer guards. A metal post can serve as a fastener 50 to attach a leg assembly.

Returning to FIGS. 1 and 2, the load-bearing exoskeleton 10 in this embodiment includes two leg assemblies 18. Each leg assembly 18 may serve as a sagittally-extending load-bearing structure that is a lower body support. Thus, in this embodiment, a supportive force is at least partially normal to an outer surface of the load-bearing structure (e.g., the outer surface where the exoskeleton interfaces with the floor). Accordingly, the applied force from the shielding garment is at least partially transmitted to the floor through the at least one lower body support.

In a preferred embodiment, the leg assembly 18 is a modified hip knee ankle foot orthosis (HKAFO). A foot platform 54 may include straps or be a slide-on type shoe to help hold a user's foot in the exoskeleton. The foot platform 54 may be rubberized or coated with another high-friction “non-slip” material. Other designs for a foot platform are certainly possible. The foot platform 54 can be connected by a first rotational joint 56 to a first structural beam 58. Knee joint 60 can connect the first structural beam 58 to a second structural beam 62. The first and second structural beams 58, 62 may have adjustable lengths, if desired. The knee joint 60 which connects the first and second structural beams 58, 62 may be a single-pivot type joint that allows for nearly full knee flexion but has a hard stop to prevent hyperextension (e.g., motion of the knee past a vertical orientation in a forward direction).

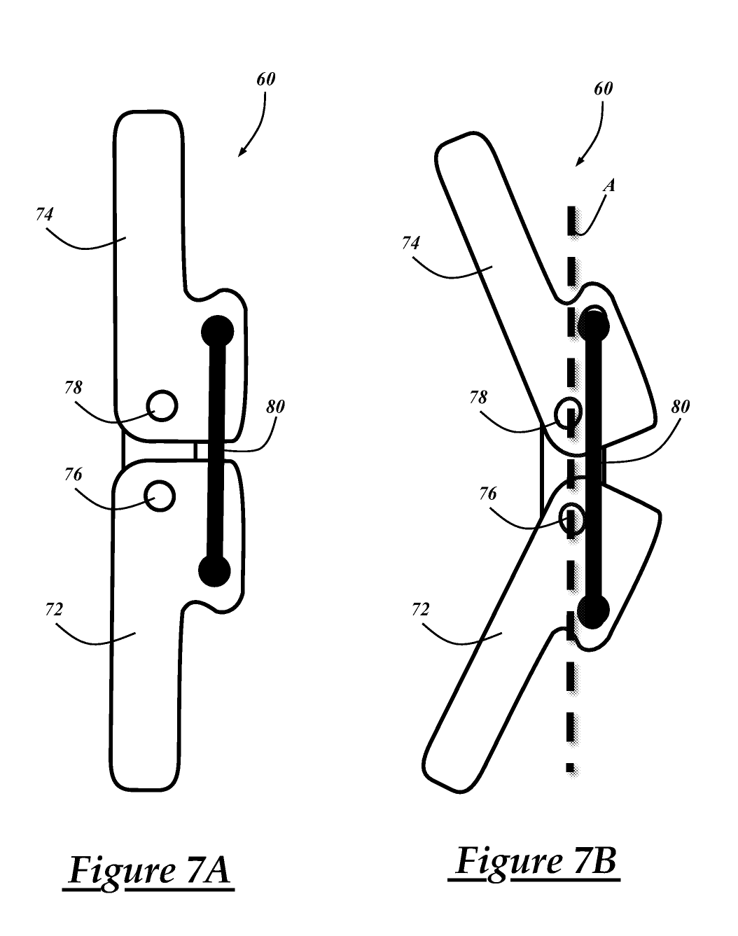

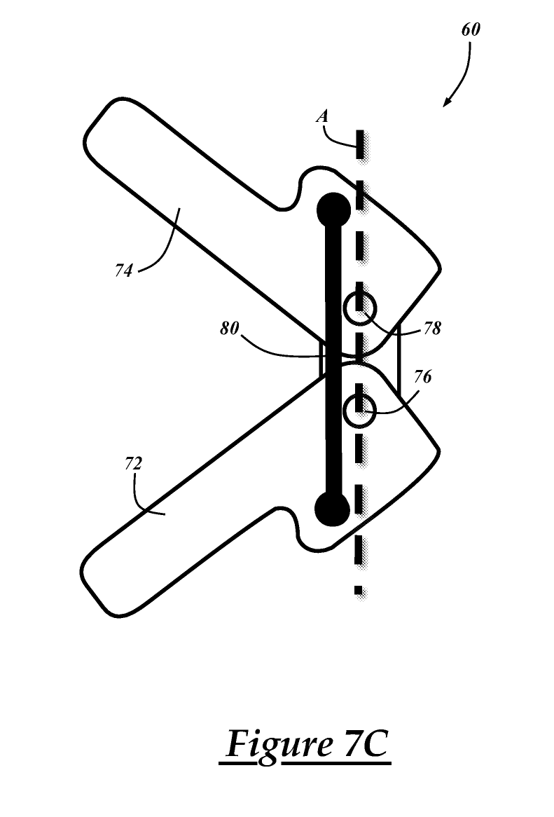

FIGS. 7A-7C schematically illustrate another embodiment of the knee joint 60 which allows for vertical transmission of load throughout a range of angles, not just at full extension. The knee joint 60 may include a lower member 72 for connecting the first structural beam (not shown in FIG. 7) and an upper member 74 for connecting the second structural beam (not shown in FIG. 7). The knee joint may include two rotational joints 76, 78 which are at least partially limited in their rotational motion by a tension element 80. The tension element 80 may include a spring, a rod, or a pneumatic cylinder, to cite a few examples. Because the tension element 80 tries to pull the joint back to full extension, the exoskeleton is still able to conduct at least some of the force down as the knee rotates around the knee pivot axis A, as shown in FIG. 7B. FIG. 7C shows an undesirable buckling point, which may be avoided by including a mechanical damper or stop to prevent rotational movement beyond a certain point (e.g. when the tension element 80 is closer to the user's knee than the knee pivot axis A). Other embodiments for the knee joint 60 besides those illustrated are certainly possible. For example, the knee joint could have a cam and follower design or any other rotational joint design that facilitates transmission of force via the structural beams of the leg assembly.

Returning to FIGS. 1 and 2, the load-bearing exoskeleton may be balanced about a second rotational joint 64 in the leg assembly 18 that is located to correspond to the greater trochanter of a user. In such an embodiment, no net rotational moment would be imposed on the user by the exoskeleton, especially when standing still. The exoskeleton may create zero net rotational moment at the hip or even induce a counter-rotational moment due to extra mass being loaded anteriorly as part of the exoskeleton itself or the attached shielding garment (e.g., a user's back muscles may not need to fully support his or her own body weight in flexion). The leg assembly 18 terminates with an attachment piece 66 that serves to attach the leg assembly to the pelvis assembly 16. The attachment piece 66 may extend above the hip joint 36 as shown, or it may only extend from the leg assembly up to the hip joint without extending beyond the hip joint. Plastic cuffs 68 may be included to help anchor the assembly to the associated leg segment (e.g., thigh or calf) and may be secured via hook-and-loop straps. Plastic cuffs 68, if included, may be located generally at the midpoint of the first and second structural beams 58, 62.

During use, the load-bearing exoskeleton 10 can vary between a plurality of states: unlocked and locked. During the unlocked state, the joints are free to rotate so that users may walk almost normally and assume whatever position they wish in order to perform procedures. Once users are in a desired posture, the device may be switched into a locked state, during which one or more joints would hold their rotational position, thereby providing a rigid support to convey most of the weight or all of the weight of the shielding garment down to the floor. In a preferred embodiment, all of the joints in the exoskeleton 10 would hold their rotational position while in the locked state. In another embodiment, only one or more leg joints (e.g., the knee joint) lock while the hip joint remains free to move so a user can flex and extend (i.e., lean forward and backward) if desired. The joints of the device could be switched between the two states through either mechanical or electromechanical means (e.g., pulleys, solenoids, etc). In one implementation, the rotational joints are passive joints with a spring powered button that would move into dents on each half of the joint once a predetermined position had been reached (i.e., vertical, full extension) and provide some resistance to movement in the joints. Motion could resume in the other direction in the joint with a higher applied torque from the leg. This implementation avoids hand actuation of buttons and/or electronics, making the exoskeleton easier to use during treatment procedures, for example.

It is to be understood that the foregoing description is of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A load-bearing exoskeleton, comprising:
a plurality of sagittally-extending load-bearing structures, wherein at least one sagittally-extending load-bearing structure is an upper body support and at least one sagittally-extending load-bearing structure is a lower body support, wherein a supportive force is provided to counteract at least some of the weight of a shielding garment, the supportive force being at least partially normal to an outer surface of at least one of the sagittally-extending load-bearing structures when an applied force from the shielding garment is encountered, the applied force from the shielding garment being at least partially transmitted to the floor through the at least one lower body support.

2. The load-bearing exoskeleton of claim 1, further comprising at least one transversely-extending load-bearing structure located between at least one upper body support and at least one lower body support.

3. The load-bearing exoskeleton of claim 2, wherein the at least one transversely-extending load-bearing structure located between the at least one upper body support and at least one lower body support further includes one or more coronally-extending load-bearing structures.

4. The load-bearing exoskeleton of claim 3, wherein at least part of the weight of at least one of the sagittally-extending load-bearing structures, at least one of the transversely-extending load-bearing structures, or a user's upper body is transmitted to the floor through the at least one lower body support.

5. A load-bearing exoskeleton, comprising:
at least one shielding garment attachment mechanism;
a pelvis assembly including one or more hip joints, the attachment mechanism being connected to and supported by the pelvis assembly; and
one or more leg assemblies attached to the pelvis assembly via the one or more hip joints, wherein the hip joint of the pelvis assembly is configured to allow for rotational movement of the leg assembly about a medial-lateral axis.

6. The load-bearing exoskeleton of claim 5, further including a thorax assembly that is pivotally mounted to the pelvis assembly and is configured to allow lateral flexing of a thorax of a user.

7. The load-bearing exoskeleton of claim 6, wherein the thorax assembly includes a plurality of shoulder extensions and a plurality of rib extensions that radiate from a thoracic nexus.

8. The load-bearing exoskeleton of claim 7, wherein the thoracic nexus is attached to the pelvis assembly via a central beam.

9. The load-bearing exoskeleton of claim 8, wherein the central beam is pivotally mounted with a rotational joint to the pelvis assembly.

10. The load-bearing exoskeleton of claim 7, wherein the thorax assembly includes an open back.

11. The load-bearing exoskeleton of claim 5, wherein the hip joint includes a mobile internal portion that attaches one of the leg assemblies to the pelvis assembly.

12. The load-bearing exoskeleton of claim 11, wherein the mobile internal portion is a cart assembly that is slidably mounted to a track assembly.

13. The load-bearing exoskeleton of claim 12, wherein the cart assembly includes one or more stabilizing components that help facilitate translation along the track assembly.

14. The load-bearing exoskeleton of claim 12, wherein the cart assembly includes one or more translation components that help decrease friction between the track assembly and the cart assembly.

15. The load-bearing exoskeleton of claim 5, wherein the attachment mechanism is connected to the pelvis assembly via the thorax assembly.

16. A load-bearing exoskeleton, comprising:
at least one shielding garment attachment mechanism;
a pelvis assembly; and
one or more leg assemblies attached to the pelvis assembly, each leg assembly including a rotational joint at a location generally corresponding to a greater trochanter of a user, wherein the rotational joint includes a locking mechanism that is configured to inhibit rotational movement of at least part of the leg assembly when the rotational joint is in a locked position.

17. The load-bearing exoskeleton of claim 16, wherein each leg assembly includes a first structural beam and a second structural beam with a knee joint located between the first and second structural beams.

18. The load-bearing exoskeleton of claim 17, wherein the knee joint includes one or more locked positions.

19. The load-bearing exoskeleton of claim 17, wherein the rotational joint and the knee joint each have one or more unlocked positions, and the rotational joint and the knee joint are configured to allow for rotational motion when in an unlocked position.

20. The load-bearing exoskeleton of claim 17, wherein each leg assembly includes a foot platform connected to the first structural beam via a rotational joint so that each leg assembly comprises a modified hip knee ankle foot orthosis (HKAFO).
